(19) United States
(12) Patent Application Publication
(10) Pub. No.: US 2008/0199439 A1
(43) Pub. Date: Aug. 21, 2008

(54) IAP-binding cargo molecules and peptidomimetics for use in diagnostic and therapeutic methods

(76) Inventor: George L. McLendon, Princeton, NJ (US)

Correspondence Address: PEPPER HAMILTON LLP, ONE MELLON CENTER, 50TH FLOOR, 500 GRANT STREET, PITTSBURGH, PA, 15219, US

(21) Appl. No.: 10/777,946

(22) Filed: Feb. 11, 2004

Related U.S. Application Data
(60) Provisional application No. 60/446,903, filed on Feb. 12, 2003.

(51) Int. Cl. A61K 48/00; G01N 33/53; C12N 5/02

(52) U.S. Cl. 424/93.7; 435/7.1; 435/375

(57) ABSTRACT

The present invention features peptides, peptidomimetics and methods of their use for binding to Inhibitor of Apoptosis Proteins (IAPs) in cells and prevents the IAP's from exerting their apoptosis-suppressing function with caspases in the cell. An IAP binding cargo molecule having a binding portion mimicking the structural and biological features of the Smac amino-terminal tetrapeptide that is capable of relieving XIAP-mediated suppression of apoptosis in mammalian cells, and a cargo portion with a functional group having a detectable property or therapeutic function is disclosed.

### CROSS REFERENCE TO RELATED APPLICATIONS

This application claims the benefit and priority of U.S. Provisional Application Ser. No. 60/446,903 filed Feb. 12, 2003 the contents of which are incorporated herein by reference in their entirety.

### BACKGROUND

Apoptosis (programmed cell death) plays a central role in the development and homeostasis of all multi-cellular organisms. Alterations in apoptotic pathways have been implicated in many types of human pathologies, including developmental disorders, cancer, autoimmune diseases, as well as neuro-degenerative disorders.

Thus, the programmed cell death pathways have become attractive targets for development of therapeutic agents. In particular, attention has been focused on anti-cancer therapies using pro-apoptotic agents such as conventional radiation and chemo-therapy. These treatments are generally believed to trigger activation of the mitochondria-mediated apoptotic pathways. However, these therapies lack molecular specificity, and more specific molecular targets are needed.

Apoptosis is executed primarily by activated caspases, a family of cysteine proteases with aspartate specificity in their substrates. Caspases are produced in cells as catalytically inactive zymogens and are proteolytically processed to become active proteases during apoptosis. In normal surviving cells that have not received an apoptotic stimulus, most caspases remain inactive. Even if some caspases are aberrantly activated, their proteolytic activity can be fully inhibited by a family of evolutionarily conserved proteins called IAPs (inhibitors of apoptosis proteins) (Deveraux & Reed, Genes Dev. 13: 239-252, 1999). Each of the IAPs contains 1-3 copies of the so-called BIR (baculoviral IAP repeat) domain and directly interacts with and inhibits the enzymatic activity of mature caspases. Several distinct mammalian IAPs including XIAP (SEQ ID NO: 1), survivin (SEQ ID NO: 2), and Livin/ML-IAP (SEQ ID NO: 3) (Kasof & Gomes, J. Biol. Chem. 276: 3238-3246, 2001; Vucic et al. Curr. Biol. 10: 1359-1366, 2000; Ashhab et al. FEBS Lett. 495: 56-60, 2001), have been identified, and they all exhibit anti-apoptotic activity in cell culture (Deveraux & Reed, 1999, supra). As IAPs are expressed in most cancer cells, they may directly contribute to tumor progression and subsequent resistance to drug treatment.

In normal cells signaled to undergo apoptosis, however, the IAP-mediated inhibitory effect is removed, a process at least in part performed by a mitochondrial protein named Smac (SEQ ID NO: 6) (second mitochondria-derived activator of caspases; Du et al. Cell 102: 33-42, 2000) or DIABLO (direct IAP binding protein with low pI; Verhagen et al. Cell 102: 43-53, 2000). Smac (SEQ ID NO: 6), synthesized in the cytoplasm, is targeted to the inter-membrane space of mitochondria. Upon apoptotic stimuli, Smac is released from mitochondria back into the cytosol, together with cytochrome c. Whereas cytochrome c induces multimerization of Apaf-1 (SEQ ID NO: 7) to activate procaspase-9 (SEQ ID NO: 8) and procaspase-3 (SEQ ID NO: 9), Smac eliminates the inhibitory effect of multiple IAPs. Smac interacts with all IAPs that have been examined to date, including XIAP (SEQ ID NO: 1), c-IAP1 (SEQ ID NO: 4), c-IAP2 (SEQ ID NO: 5), and survivin (SEQ ID NO: 2) (Du et al., 2000, supra; Verhagen et al., 2000, supra). Thus, Smac (SEQ ID NO: 6) appears to be a master regulator of apoptosis in mammals.

Similar to mammals, flies contain two IAPs, DIAP1 (SEQ ID NO: 10) and DIAP2 (SEQ ID NO: 11), that bind and inactivate several Drosophila caspases (Hay, Cell Death Differ. 7: 1045-1056, 2000). DIAP1 (SEQ ID NO: 10) contains two BIR domains; the second BIR domain (BIR2) is necessary and sufficient to block cell death in many contexts. In Drosophila cells, the anti-death function of DIAP1 (SEQ ID NO: 10) is removed by three pro-apoptotic proteins, Hid (SEQ ID NO: 12), Grim (SEQ ID NO: 13), and Reaper (SEQ ID NO: 14), which physically interact with the BIR2 domain of DIAP1 (SEQ ID NO: 10) and remove its inhibitory effect on caspases. Thus Hid (SEQ ID NO: 12), Grim (SEQ ID NO: 13), and Reaper (SEQ ID NO: 14), represent the functional homologs of the mammalian protein Smac (SEQ ID NO: 6). However, except for their N-terminal 10 residues, Hid (SEQ ID NO: 12), Grim (SEQ ID NO: 13), and Reaper (SEQ ID NO: 14), share no sequence homology with one another, and there is no apparent homology between the three Drosophila proteins and Smac (SEQ ID NO: 6).

In commonly-owned co-pending application Ser. No. 09/965,967 (the entirety of which is incorporated by reference herein), it was disclosed that the above described biological activity of Smac (SEQ ID NO: 6) is related to binding of its N-terminal four residues to a featured surface groove in a portion of XIAP (SEQ ID NO: 1) referred to as the BIR3 (SEQ ID NO: 15) domain. This binding prevents XIAP (SEQ ID NO: 1) from exerting its apoptosis-suppressing function in the cell. It was further disclosed that N-terminal tetrapeptides from IAP binding proteins of the Drosophila pro-apoptotic proteins Hid (SEQ ID NO: 12), Grim (SEQ ID NO: 13) and Veto (SEQ ID NO: 14) function in the same manner.

Commonly-owned co-pending International Application No. PCT/US02/17342, filed May 31, 2002, discloses assays for use in high throughput screening or rational drug design of agents that can mimic the activity of Smac tetrapeptide or its homologs by binding to a BIR domain of an IAP, thereby relieving IAP-mediated suppression of apoptosis. The assays utilize a labeled mimetic of an IAP-binding tetrapeptide that binds to the appropriate BIR domain (preferably BIR3 (SEQ ID NO: 15)), wherein at least one measurable feature of the label changes as a function of the mimetic being bound to the IAP or free in solution. The BIR domain of an IAP is contacted with the labeled mimetic to form a complex, and the complex is exposed to a compound to be tested for BIR binding. Displacement of the labeled mimetic from the complex, if any, by the test compound, is measured. The labeled mimetic may be derived from any of the IAP-binding peptides disclosed in either U.S. application Ser. No. 09/965,967 or PCT/US02/17342, one example being the tetrapeptide AVPC (SEQ ID NO: 17) linked to a badan dye.

### SUMMARY

The present invention relates to the field of screening, diagnosis and treatment of cell proliferative disease. Specifically, the invention features molecules which have a targeting agent portion that binds to IAPs including but not limited to XIAP (SEQ ID NO: 1), c-IAP1 (SEQ ID NO: 4), c-IAP2 (SEQ ID NO: 5), and survivin in cells, and a cargo portion that can include therapeutic or diagnostic functionality. The molecules of the present invention include peptidomimetics, peptides, and polypeptides of such IAP binding cargo molecules that permeate the cell membrane illustrated schematically by the structure of formula (I).

IAP Binding-Cargo (I)

According to one aspect of the invention, a method of selectively identifying neoplastic or cancer cells in a mixed population of cells is provided. The method comprises contacting the mixed cell population with a cell permeant IAP-binding cargo molecule under conditions enabling the IAP-binding cargo molecule to bind IAP within the neoplastic cells, thereby selectively identifying the neoplastic cells by a detectable change in a property of the cargo molecule. The cells may be cultured cells or primary cells from a patient (human or animal). Alternatively, the cells may be present within the patient, and the contacting accomplished by introducing the IAP-binding cargo molecule into the patient.

In an embodiment of the IAP-binding cargo molecule, the cargo portion of the molecule comprises a dye label. In other embodiments, the cargo portion of the molecule can be but is not limited to an NMR-active nucleus or an MRI contrast agent, and the selective identification is performed through nuclear magnetic resonance or magnetic resonance imaging. Alternatively, the labeled IAP-binding cargo molecule comprises a radioisotope and the selective identification is performed through positron emission tomography.

Another aspect of the invention features a method of selectively damaging or inducing apoptosis in neoplastic cells killing neoplastic cells in a mixed population of cells. The method includes contacting a sample of the mixed cell population with an IAP-binding cargo molecule. The cargo portion of the molecule is directly or indirectly toxic to cells (e.g., a radioisotope or a photosensitizing agent. The IAP binding portion of the molecule binds to IAP within the neoplastic cells, whereupon the toxic agent of the cargo directly or indirectly exerts its toxic effect, thereby damaging or killing the neoplastic cells.

Another embodiment of the present invention is a composition that includes cells and an IAP binding cargo molecule. The IAP binding cargo molecule binds to an IAP protein, including XIAP (SEQ ID NO: 1), c-IAP1 (SEQ ID NO: 4), c-IAP2 (SEQ ID NO: 5), and survivin (SEQ ID NO: 2), preferably it binds to a BIR surface groove of an IAP protein, and even more preferably the molecule binds to the BIR3 (SEQ ID NO: 15) surface groove of an XIAP protein. The IAP binding cargo molecule permeates into the cells and can displace an IAP protein from a caspase in the cells. The cargo portion of an IAP binding cargo molecule preferably has a detectable property which is modified upon chemical interaction of the molecule with the IAP protein in the cells. This composition is useful as a control for monitoring the presence of IAP in the cells undergoing treatment or for use as a standard in the detection of abnormal IAP levels in a sample of cells. The detectable property may be the emission of light by the cargo portion of the molecule which changes when the IAP bonding portion of the molecule binds to an IAP protein. The IAP binding cargo molecule can be a tetrapeptide of structure (II):

Xₐₐ₁—Xₐₐ₂—Xₐₐ₃—Xₐₐ₄-cargo (II)

wherein X_{aaN} are amino acids the make up the IAP binding portion of the molecule that chemically bonds to an IAP protein, and the cargo portion of the molecule includes but is not limited to fluorogenic or chromogenic groups. The one or more cells in the composition may include but are not limited to cells from a bodily fluid, tissue, tumor, fibroid, neoplastic cells, nervous system cells or any combination of these. The IAP bonding cargo molecule may be an NMR-active nucleus or an MRI contrast agent and the selective identification of the cargo portion of the molecule performed through nuclear magnetic resonance or magnetic resonance imaging. An example of a preferred IAP binding portion of the molecule is illustrated by the tetrapeptide AVPC (SEQ ID NO: 17 ), linked to the fluorogenic dye badan to form the IAP binding cargo molecule (AVPC-badan).

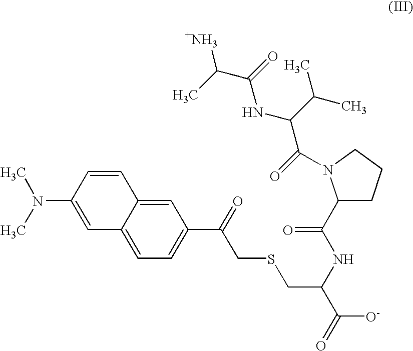

Another embodiment of the present invention is a method of identifying IAP in cells that includes monitoring a mixture of one or more IAP binding cargo molecules with one or more sample cells for a change in a detectable property of one or more of the IAP binding cargo molecules in the mixture. The detectable property of the cargo molecule changes upon formation of a complex between the IAP binding cargo molecule and IAP protein, including XIAP (SEQ ID NO: 1), c-IAP1 (SEQ ID NO: 4), c-IAP2 (SEQ ID NO: 5), and survivin (SEQ ID NO: 2) in the sample cells; the IAP may be bound to a caspase within the cell. Monitoring may be performed on cells and an IAP binding cargo molecule in a fluid sample, a flowing fluid, or fluids following purification. This invention may be used to detect abnormal expression, over or under expression, of IAP in cells and begin a course of treatment of the cells. Preferably the IAP binding cargo molecule is used to detect overexpression of IAP in cells. The method may further include the act of comparing a change in a detectable property of one or more IAP binding cargo molecules mixed with one or more control cells to the detectable change in the property of the one or more IAP binding cargo molecules mixed with one or more sample cells. The comparison may be related to the amount of IAP in the sample cells. The method may include the act of combining one or more IAP binding cargo molecules with one or more cells including but not limited to sample cells, control cells, or various combination of these cells. The monitoring may use the absorption or emission of radiant energy by the mixture of IAP and the cells, including but not limited to magnetic resonance, fluorescence, chemiluminesnce, magnetic resonance imaging, and positron emission tomography. Preferably the change in the detectable property of one or more of the IAP binding cargo molecules in the mixture chemically binding to the IAP in the cells is a change in the intensity of fluorescent emission of said IAP binding molecule. Even more preferably a change occurs in the fluorescent emission of one or more IAP binding cargo molecules capable of displacing IAP from a caspases in the sample cells. An example of an IAP bonding cargo molecule used in the method is AVPC-badan.

A method of treating cells of the present invention includes identifying the expression of IAP in cells and administering an amount of a cell permeant IAP-binding cargo molecule or other therapeutic to said cells to modify the amount of IAP in the cells. For example, following identification of higher than normal levels of IAP in a sample of cells, optionally by comparison to a control sample of cells, purified Smac, a peptidomimetic of an IAP binding protein, or an IAP binding cargo molecule may be added to the cells to induce apoptosis. This invention can be used to identify cells in need of treatment, treat the cells, and monitor the progress of the treatment of the cells having the abnormal IAP levels. The act of identifying cells having abnormal IAP expression includes monitoring a mixture of one or more IAP binding cargo molecules with one or more sample cells for a change in a detectable property of one or more of the IAP binding cargo molecules. The detectable property changes upon formation of a complex between the IAP binding molecule and IAP in said sample cells.

Another embodiment of the present invention is an article that includes packaging material containing a composition of an IAP binding cargo molecule. The packaging material has a label that indicates how the IAP binding cargo composition can be used for detecting levels of IAP in a sample of cells. The label may further indicate how the IAP binding cargo molecule composition can be used to treat cells where an abnormal level of IAP expression is determined.

An embodiment of the present invention is a method of selectively identifying neoplastic cells in a mixed population of cells. In the method a sample of the mixed cell population is contacted with one or more IAP-binding cargo molecules that may have an IAP binding portion of the molecule that is a peptide, or peptidomimetic under conditions enabling the IAP-binding cargo molecule to bind IAP within the neoplastic cells and thereby selectively identifying the neoplastic cells. The cells may include but are not limited to cultured cells, cells are removed from a subject by biopsy, or cells from a fluid. The contacting may be performed by introducing the labeled IAP-binding cargo molecule into a living subject possessing or suspected of possessing the neoplastic cells. The IAP-binding cargo molecule can have a dye label cargo portion and preferably the dye is a fluorogenic dye. The labeled IAP-binding cargo molecule may have an NMR-active nucleus or a contrast agent and the selective identification is performed through nuclear magnetic resonance or magnetic resonance imaging. The labeled IAP-binding cargo molecule may have a cargo portion of the molecule that is a radioisotope and where the selective identification performed through positron emission tomography.

Another embodiment of the present invention is a method of selectively damaging or killing neoplastic cells in a mixed population of normal and neoplastic cells. The method includes contacting a sample of the mixed cell population with a cell permeant IAP-binding cargo molecule wherein the cargo portion of the molecule is an agent that is directly or indirectly toxic to cells. Under conditions enabling the IAP-binding cargo molecule to bind IAP within the neoplastic cells, the agent directly or indirectly exerts its toxic effect, thereby damaging or killing the neoplastic cells. The method may use an agent that is a radioisotope. The method may use a photosensitizing agent and the selective damaging or killing is performed by exposing the cell population to light.

Other features and advantages of the invention will be understood by reference to the drawings and detailed description that follow.

### DESCRIPTION OF THE DRAWINGS

The file of this patent contains at least one drawing/photograph executed in color. Copies of this patent with color drawing(s)/photograph(s) will be provided by the Office upon request and payment of the necessary fee.

In part, other aspects, features, benefits and advantages of the embodiments of the present invention will be apparent with regard to the following description, appended claims and accompanying drawings where:

FIG. 1 HeLa cells are exposed to AVPC-badan (0.1 mM) in the presence of (A) 0 equivalents of AVPF (SEQ ID NO: 19) where light ring at the outer edge of the cells is AVPC-badan binding to IAP within cells, and (B) 100 equivalents of the competitor, AVPF (SEQ ID NO: 19). The emission between 435-485 nm after exposure of cells for 30 minutes is shown in each case. (C) A498 cells are exposed to AVPC-badan (8 mM) in the presence of 0 and 100 equivalents of the competitor, AVPF (SEQ ID NO: 19). The emission between 385-470 nm and >505 nm after exposure of cells for 36 minutes is shown in each case;

FIG. 2 shows Confocal microscope images of HeLa cells loaded with AVPC-badan (17 mM) after 48 minutes. (a) emission observed between 385-470 nm. (b) emission observed with a 505-550 nm bandpass filter (c) transmitted white light image and (d) composite of a, b and c. In (d), the contrast between the background region and the cytosol of cells loaded with AVPC-badan (yellow or light ring near outer edge of cells in composite image) is dramatic. In HeLa cells, where XIAP (SEQ ID NO: 1) expression is high and enhancement of the dye's emission upon AVPC-badan:XIAP binding is expected to be large;

FIG. 3 shows confocal microscope images of MCF7 cells loaded with AVPC-badan (17 mM) after 48 minutes. (a) emission observed between 385-470 nm. (b) emission observed with a 505-550 nm bandpass filter (c) transmitted white light image and (d) composite of a, b and c. In (d), the contrast between the background region and the cytosol loaded with AVPC-badan is minimal in MCF-7 cells where XIAP (SEQ ID NO: 1) expression is low;

FIG. 4 is a table of tetrapeptides which may be used to form the IAP binding portion of an IAP binding cargo molecule of the present invention, numbers to the right of each sequence in parentheses are SEQ ID NOs.

### DETAILED DESCRIPTION

Before the present compositions and methods are described, it is to be understood that this invention is not limited to the particular molecules, compositions, methodologies or protocols described, as these may vary. It is also to be understood that the terminology used in the description is for the purpose of describing the particular versions or embodiments only, and is not intended to limit the scope of the present invention which will be limited only by the appended claims.

It must also be noted that as used herein and in the appended claims, the singular forms “a”, “an”, and “the” include plural reference unless the context clearly dictates otherwise. Thus, for example, reference to a “cell” is a reference to one or more cells and equivalents thereof known to those skilled in the art, and so forth. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference. Nothing herein is to be construed as an admission that the invention is not entitled to antedate such disclosure by virtue of prior invention.

The present invention features peptides, peptidomimetics and methods of their use for binding to Inhibitor of Apoptosis Proteins (IAPs), including but not limited to XIAP (SEQ ID NO: 1), c-IAP1 (SEQ ID NO: 4), c-IAP2 (SEQ ID NO: 5), survivin (SEQ ID NO: 2), and DIAP (SEQ ID NO: 10-11). The cellular function of IAPs is to suppress programmed cell death, whereas Smac and other IAP binding proteins relieve that suppression. The mammalian IAP binding protein Smac is dependent upon binding of its N-terminal four residues to a featured surface groove in a portion of XIAP referred to as the BIR3 (SEQ ID NO: 15) domain. This binding prevents XIAP from exerting its apoptosis-suppressing function with caspases in the cell. An IAP binding cargo molecule comprising or mimicking the structural and biological features of the Smac amino-terminal tetrapeptide is capable of relieving XIAP-mediated suppression of apoptosis in mammalian cells and providing a functional group having a detectable property or therapeutic to the cell.

The terms “mimetic”, “peptide mimetic” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically modified peptides, as well as non-peptide agents such as small molecule drug mimetics, as further described below. Knowing these precise structural features of naturally-occurring IAP-binding cargo molecules, it is advantageous, and well within the level of skill in this art, to design peptidomimetics that have an equivalent structure or function. Such mimetics are another feature of the present invention. Mimetics of the core IAP-binding tetrapeptides are preferred in this aspect of the invention. The tetrapeptide is suitably small, and its structural features in relation to the IAP binding groove are well characterized, thereby enabling the synthesis of a wide variety of mimetic compounds. An added advantage of compounds of this size include improved solubility in aqueous solution, cell permeance, and ease of delivery to selected targets in vivo.

In one embodiment, the IAP-binding cargo molecules of the invention are modified to produce peptide mimetics by replacement of one or more naturally occurring side chains of the 20 genetically encoded amino acids (or D amino acids) with other side chains, for instance with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclics. For example, proline analogs can be made in which the ring size of the proline residue is changed from 5 members to 4, 6, or 7 members. Cyclic groups can be saturated or unsaturated, and if unsaturated, can be aromatic or non-aromatic. Heterocyclic groups can contain one or more nitrogen, oxygen, and/or sulphur heteroatoms. Examples of such groups include the furazanyl, fliryl, imidazolidinyl, imidazolyl, imidazolinyl, isothiazolyl, isoxazolyl, morpholinyl (e.g. morpholino), oxazolyl, piperazinyl (e.g. 1-piperazinyl), piperidyl (e.g. 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g. 1-pyrrolidinyl), pyrrolinyl, pyrrolyl, thiadiazolyl, thiazolyl, thienyl, thiomorpholinyl (e.g. thiomorpholino), and triazolyl. These heterocyclic groups can be substituted or unsubstituted. Where a group is substituted, the substituent can be alkyl, alkoxy, halogen, oxygen, or substituted or unsubstituted phenyl. Peptidomimetics may also have amino acid residues that have been chemically modified by phosphorylation, sulfonation, biotinylation, or the addition or removal of other moieties.

A variety of techniques are available for constructing peptide mimetics with the same or similar desired biological activity as the corresponding native but with more favorable activity than the peptide with respect to solubility, stability, cell permeability, and/or susceptibility to hydrolysis or proteolysis (see, e.g., Morgan & Gainor, Ann. Rep. Med. Chem. 24, 243-252, 1989). Certain peptidomimetic compounds are based upon the amino acid sequence of the peptides of the invention. Often, peptidomimetic compounds are synthetic compounds having a three-dimensional structure (i.e. a “peptide motif”) based upon the three-dimensional structure of a selected peptide. The peptide motif provides the peptidomimetic compound with the desired biological activity, i.e., binding to IAP, wherein the binding activity of the mimetic compound is not substantially reduced, and is often the same as or greater than the activity of the native peptide on which the mimetic is modeled. Peptidomimetic compounds can have additional characteristics that enhance their therapeutic application, such as increased cell permeability, stability to radiological elements, greater affinity and/or avidity and prolonged biological half-life.

Peptidomimetic design strategies are readily available in the art (see, e.g., Ripka & Rich, Curr. Op. Chem. Biol. 2, 441-452, 1998; Hruby et al., Curr. Op. Chem. Biol. 1, 114-119, 1997; Hruby & Balse, Curr. Med. Chem. 9, 945-970, 2000). One class of peptidomimetics a backbone that is partially or completely non-peptide, but mimics the peptide backbone atom-for atom and comprises side groups that likewise mimic the functionality of the side groups of the native amino acid residues. Several types of chemical bonds, e.g. ester, thioester, thioamide, retroamide, reduced carbonyl, dimethylene and ketomethylene bonds, are known in the art to be generally useful substitutes for peptide bonds in the construction of peptidomimetics. Another class of peptidomimetics comprises a small non-peptide molecule that binds to another peptide or protein, but which is not necessarily a structural mimetic of the native peptide. Yet another class of peptidomimetics has arisen from combinatorial chemistry and the generation of massive chemical libraries. These generally comprise novel templates which, though structurally unrelated to the native peptide, possess necessary functional groups positioned on a nonpeptide scaffold to serve as “topographical” mimetics of the original peptide (Ripka & Rich, 1998, supra).

Peptide components of this invention preferably include the 20 naturally-occurring amino acids. However, incorporation of known artificial amino acids such as beta or gamma amino acids and those containing non-natural side chains, and/or other similar monomers such as hydroxyacids are also contemplated, with the effect that the corresponding peptide is not completely inhibited from binding IAP proteins, preferably binding the BIR3 (SEQ ID NO: 15) domain of an IAP, and being permeable to the cell. A non-limiting example includes the use of Abu, 2-aminobutyric acid as an amino acid in the IAP binding peptide AbuVPI (SEQ ID NO:23)

Because caspases are cytosolic enzymes, diagnostic, imaging, and therapeutic compounds that chemically bind with the IAP proteins cross cell membranes. The cell membrane-permeant basic peptide component of the complexes of the present invention can comprise any amino acid sequence that confers the desired intracellular translocation and targeting properties to the IAP binding cargo molecules. Preferably, these amino acid sequences are characterized by their ability to confer transmembrane translocation and internalization of a complex IAP binding cargo molecule construct when administered to the external surface of an intact cell, tissue or organ. The IAP binding cargo molecules permeate the cell and can be localized within cytoplasmic and/or nuclear compartments as could be demonstrated by a variety of detection methods such as, for example, fluorescence microscopy, confocal microscopy, electron microscopy, autoradiography, or immunohistochemistry.

IAP binding cargo molecules, labeled IAP binding peptides, labeled binding molecule are used interchangeably herein to refer to IAP binding cargo molecules having an overall structure illustrated schematically by formula (I):

IAP Binding-Cargo (I)

wherein the IAP binding cargo molecule may for illustrative purposes be arbitrarily divided to include an IAP binding portion and a cargo portion. The IAP binding portion of the molecule may be a peptide, peptidomimetic, or a portion of a molecule that chemically binds to an IAP protein, preferably the BIR surface groove of an IAP protein and more preferably the BIR3 (SEQ ID NO: 15) surface groove of XIAP (SEQ ID NO: 1). The IAP binding portion of the molecule chemically mimics the N-terminal tetrapeptide of Smac (SEQ ID NO: 6) and its functional homologs in other species. The cargo portion of the molecule is chemically connected to the IAP portion of the molecule and may include but is not limited to structures for imaging, therapeutics, probes, labels, or markers. The cargo portion and IAP binding portion of the molecule may be connected by a chemical bond to the IAP binding portion including but not limited to amide, ester, or disulfide bonds or by a linking group such as diaminobutane or ethylene glycol where it is desirable to separate the IAP binding portion of the molecule from the cargo portion of the molecule. The binding portion confers target protein specificity to the molecule and the cargo portion provides a functional group to the molecule for monitoring or evaluating the location of the molecule or providing a therapeutic to that location within a cell sample or a tissue in a mammal. The cargo portion of the molecule may be bonded to any portion of the IAP bindin portion of the molecule and while chemical interaction between the IAP binding portion and the cargo portion of the molecule may occur, the molecule is made so that the molecule's cell permeance, its IAP binding property, and function of the cargo portion are not adversely affected by their combination. The suitability of any IAP binding cargo molecule made by the method disclosed may be tested against AVPC-badan in cells such as HeLa known to overexpress IAP. The IAP binding molecules of the present invention are capable of permeating cells of interest, binding to IAP in the cells, and delivering the cargo to the cells.

The IAP binding cargo molecule can bind to IAP in the cell or competitively displace IAP bonded to a caspase in the cells. The IAP binding cargo molecule chemically bonds or binds to an IAP protein and displaces it from the caspase. The chemical bond between the IAP binding portion of the IAP binding cargo molecule and the IAP protein as known to those skilled in the art involves the chemical interaction between the IAP binding cargo molecule and the surface groove of the IAP protein.

Examples of IAP binding cargo molecules include those molecules used in the assays described in co-pending International Application PCT/US02/17342 the contents of which are incorporated herein by reference in their entirety. These IAP binding cargo molecules utilized a labeled mimetic of the N-terminal tetrapeptide of Smac and its functional homologs in other species and may be represented by structures of formula (IV):

Xₐₐ₁(Xₐₐ)_{Y}X_{aa(Y+1)}-cargo (IV)

wherein the cargo portion of the molecule can be therapeutic, a label, or probe moiety. The amino acids X_{aa(1 . . . Y+1)} may be any amino acid or peptidomimetic and Y is chosen so that a molecule of formula (IV) is cell permeant, displaces IAP protein from caspases, binds to an IAP protein in cells, and may be linked to a cargo portion which may be a fluorophore, radioisotope, therapeutic, or probe. Preferably Xₐₐ₁ is Ala or Abu. For example a molecule of formula (IV) may include but is not limited to the peptide AVPIAQKSE (SEQ ID NO: 49) which may be linked to the fluorophore badan to form the IAP binding cargo molecule AVPIAQKSE-badan. Treating cells known to express high levels of IAP, such as HeLA cells, with a molecule having a structure of formula (IV) can be used to determine the cell permeance, change in detectable property, and IAP binding of such molecules.

Preferably the IAP binding cargo molecule has a cargo portion that is a fluorophore attached to a tetrapeptide illustrated by structure (V):

Xₐₐ₁—Xₐₐ₂—Xₐₐ₃—Xₐₐ₄-fluorophore (V)

wherein X_{anN} may be any amino acid or peptidomimetic in the tetrapeptide that is cell permeant portion, forms an IAP binding portion in cells and displaces IAP from caspases, and is bonded to a fluorophore. Where the molecule of formula (V) includes amino acids, it is preferred that the N-terminal amino acid Xₐₙ₁ is Ala or Abu.

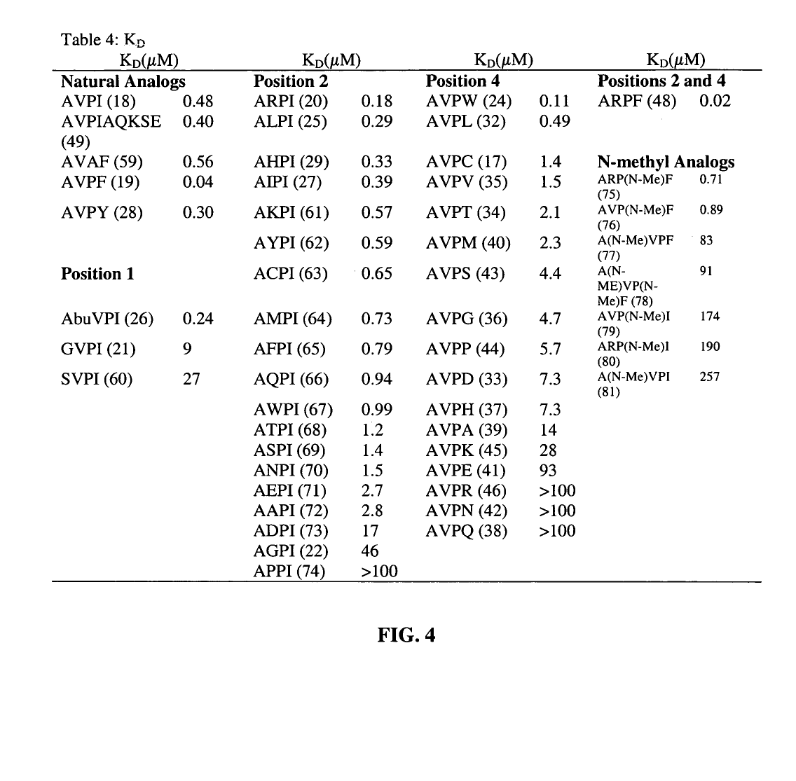

Molecules of formula (V) preferably have an IAP binding portion that include those with tetrapeptides which can displace AVPC-badan from its complex with BIR3 (SEQ ID NO: 15), and preferably those tetrapeptides having a K_{D} for the displacement of AVPC-badanadan from its complex with BIR3 complex of less than about 2. Various tetrapeptides are listed in the Table in FIG. 4. Exemplary tetrapeptides for the IAP binding portion of the molecule of the present invention include but are not limited to AVP1 (SEQ ID NO: 18), AVAF (SEQ ID NO: 59); AVPF (SEQ ID NO: 19); AVPY (SEQ ID NO: 28); AbuVPI (SEQ ID NO: 26); ARPI (SEQ ID NO: 20); ALPI (SEQ ID NO: 25); AHPI (SEQ ID NO: 29); AIPI (SEQ ID NO: 27); AVPW (SEQ ID NO: 24); AVPL (SEQ ID NO: 32); and ARPF (SEQ ID NO: 48). An example of a preferred IAP binding portion of the molecule is illustrated by the tetrapeptide AVPC (SEQ ID NO: 17), linked to the fluorogenic dye badan to form the IAP binding cargo molecule (AVPC-badan).

Without wishing to be bound by theory, upon binding to an IAP protein, the labeled IAP-binding cargo molecule packs into the groove of the BIR3 (SEQ ID NO: 15) causing a detectable shift in emission maximum and intensity when the environment of the fluorophore changes from water to the hydrophobic pocket of the protein. In the case of AVPC-badan, the emission intensity of the 550 nm peak shifts to 542 nm and increases in intensity as AVPC-badan binds to recombinant XIAP-BIR3.

It will be understood by those of skill in the art that, though the AVPC-badan dye system described herein is exemplified and preferred for practice of the invention, various IAP-binding cargo molecules or mimetics and detectable cargo moieties may be used interchangeably to create a variety of labeled compounds. Particular reference is given to the consensus tetrapeptide set forth in co-pending U.S. application Ser. No. 09/965,967, which is A-(V/T/I)-(P/A)-(F/Y/I/V), (SEQ ID Nos 18, 19, 27, 28, 30, 31, 35, 47, 50-59, 68, and 82-86), as well as to the variety of IAP-binding cargo molecules and mimetics set forth in PCT/US02/17342 the contents of which are included herein by reference in their entirety. The term “labeled IAP-binding peptide” or more generally IAP-binding cargo molecule as used herein encompasses any combination of peptides or mimetics thereof, and detectable labels customarily used in conjunction with the labeling of such molecules.

The labeled IAP-binding cargo molecule may comprise any suitable detectable label, including fluorophores, chromophores, fluorescent nanoparticles, and other dyes, isotopes, radioisotopes, metals, small molecules and the like, provided that the label when bonded to the IAP binding portion of the molecule, the label does not interfere substantially with the cell permeance or binding of the molecule to IAP . In selecting a label, preferably a detectable property of the label changes with the binding of the label to an IAP protein. The detectable property of the label may change because the interaction of the label with the cellular environment changes when the molecule binds to IAP thereby enhancing or diminishing the property. As noted earlier, a particularly suitable dye is 6-bromoacetyl-2-dimethylaminonaphthalene (badan or “b”) dye. Badan is a fluorogenic dye whose sensitivity to environmental changes has previously been made use of to probe protein binding interactions (Boxrud et al. J. Biol. Chem. 275: 14579-14589, 2000; Owenius et al., Biophys. J. 77: 2237-2250, 1999; Hiratsuka, T. J. Biol. Chem. 274: 29156-29163, 1999)

In the context of the present invention, a fluorogenic dye compound forming a portion of an IAP binding cargo molecule, undergoes a detectable change in its fluorescent signal on interaction with IAP proteins. Fluorogenic dyes suitable as the cargo portion of molecules for use in the present invention have a detectable fluorescent signal prior to the IAP binding cargo molecule interacting with an IAP protein in a cell; the fluorogenic dyes have a measurably different fluorescent signal after the IAP binding cargo molecules have reacted with the IAP protein. The fluorophore may include but is not limited to Badan, (6-bromoacetyl-2-dimethyl-aminonaphthalene; BODIPY, (N-(4,4-difluoro-1,3,5,7-tetramethyl-4-bora-3a)); acrylodan, 6-acryloyl-2-dimethylaminonaphthalen; AANS, 2-((4″iodoacetamido)anilino)naphthalene-6-sulfonic acid; and Resazurin, 7-hydroxy-3H-phenoxazin-3-one 10-oxide. Fluorogenic Dyes are either commercially available (for example, Resazurin is available as Resazurin, sodium salt, from Aldrich) or these fluorogenic dyes are capable of being synthesized using procedures reported in the literature.

Positron emission tomography (PET) is a technique for measuring the concentrations of positron-emitting radioisotopes within the tissue of living subjects. In the context of the present invention, a radionuclide may form the cargo portion of an IAP binding cargo molecule. Radionuclides suitable as the cargo portion of molecules for use in the present invention may include but are not limited to positron-emitting radionuclides which have short half-lives and high radiation energies compared with radioisotopes generally used in biomedical research. The main positron- emitting radionuclides used in PET include Carbon-11, Nitrogen-13, Oxygen-15, and Fluorine-18, with half-lives of 20 min, 10 min, 2 min, and 110 min respectively. IAP binding cargo molecules having a cargo portion that is a positron- emitting radionuclide may be administered to cells or the subject of study and an image of the distribution of the positron activity as a function of time by emission tomography made. Where the IAP binding cargo molecules interact with IAP in cells, a signal localized to these cells may be detected by emission tomography. Methods for making stable IAP binding cargo molecules with the cargo portion of the molecule being a radioisotope by labeling the peptide with a radioisotope and alternatively contacting the radiolabeled peptide with a protective coating are provided in U.S. Pat. No. 6,338,835 the contents of which are incorporated herein by reference in their entirety.

IAP-binding cargo molecules may be labeled with NMR active nuclei, including ¹³C and ¹⁹F, for use in clinical diagnosis via NMR and MRI. Likewise, the compounds may be labeled with Gd³⁺ as target-specific MRI contrast agents for cells and tissues that overproduce IAP, particularly cancer cells. Water-soluble carboxypolysaccharide coated magnetic iron oxide particles of small diameter composed of a water-soluble carboxypolysaccharide and a magnetic iron oxide having a core diameter ranging from about 2 to about 7 nm may be used as MRI or X-ray contrast agents for the cargo portion of the molecule as disclosed in U.S. Pat. No. 5,766,572 the contents of which are incorporated herein by reference in their entirety. The carboxypolysaccharide includes polysaccharides obtained by converting the reducing end group of a water-soluble polysaccharide into a carboxyl group. Reactions for coupling such carboxy terminated polysaccharide coated magnetic nanoparticles with an IAP binding portion of molecules of the present invention may performed using techniques known to those skilled in the chemical arts.

IAP-binding cargo molecules will also find utility as therapeutic agents. For instance, an IAP binding cargo molecule where the cargo portion is radiolabeled may be used for radiation therapy. Through the use of these cargo molecules, radioactive atoms may be administered to a tumor or other population of cancer cells that overexpress IAP protein. The IAP in the tumor becomes bonded to the radionucleide of the IAP binding cargo molecule. Similarly, IAP-binding cargo molecules may be designed to incorporate a dye that is active in photodynamic therapy. Other such therapeutic utilities will be apparent to those skilled in the art.

Cells may be mixed and optionally incubated with an IAP binding cargo molecule in a fluid sample in a vessel or wells, a flowing fluid, or fluids following purification. These samples may be monitored for changes in a detectable property. For example, flow cytometry is a method for analyzing cells labeled with a fluorescent probe molecule on a flow cytometer. In a flow cytometer the cells pass single-file through a focused laser beam where they emit fluorescence from the probe within the cell that can be detected by the photomultiplier tubes of the cytometer. Cells with abnormal expression, high or low, of IAP may be contacted and optionally incubated with IAP binding cargo molecules having a fluorescent probe. The binding of the IAP bonding cargo molecules to the IAP protein in the cells may be detected by the flow cytometer. The intensity of the fluorescence emission can be measured, digitized, and stored on a computer disk for analysis and comparison to the fluorescent emission from control cells, samples of cells being treated, or other cell samples whose IAP expression is to be determined.

The invention also provides a method of screening for IAP proteins in cells with a molecule that binds the surface groove of the BIR domain in an IAP protein. The method includes combining a synthetic IAP binding cargo molecule and the IAP proteins from cells, under conditions wherein in the IAP binding cargo molecule and IAP protein can combine. It may include the step of incubating a sample of cells with an IAP binding cargo molecule. IAP binding by the molecule, an indication of the presence of IAP in the cells, may be determined by monitoring a detectable binding property of the IAP binding cargo molecule. A change in the detectable property of the IAP binding molecule may be used to determine the expression of IAP in the cells. Where IAP protein is over expressed in cells, the IAP binding cargo molecule binds the IAP and relieves IAP-mediated inhibition of caspase activity in the cell.

The IAP-binding cargo molecules may be utilized in various assays to screen for and identify compounds capable of acting as agonists or antagonists of the IAP-caspase protein interactions within cells. IAP binding cargo molecules which disrupt IAP-caspase interaction, antagonists of this interaction, are expected to be useful as pro-apoptotic drugs for treatment of cell proliferative diseases such as cancer. Agonists of this interaction are expected to be useful as anti-apoptotic drugs for treatment of diseases where inhibition of apoptosis is needed, e.g., degenerative diseases such as Alzheimer's disease.

As used herein, the term “pharmaceutically acceptable salt” refers to those salts of the IAP binding cargo molecules and peptidomimetics thereof which retain the biological effectiveness and properties of the free bases or free acids, cell permeation and IAP binding, and which are not biologically or otherwise undesirable. If the compound exists as a free base, the desired salt may be prepared by methods known to those of ordinary skill in the art, such as treatment of the compound with an inorganic acids such as hydrochloric acid, hydrobromic acid, sulfuric acid, nitric acid, phosphoric acid and the like; or with an organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. If the compound exists as a free acid, the desired salt may also be prepared by methods known to those of ordinary skill in the art, such as the treatment of the compound with an inorganic base or an organic base. Salts derived from inorganic bases include, but are not limited to, the sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like.

The peptidomimetic, specific binding agent, or the polypeptide of the present invention may include solvent molecules within their crystal lattice. Such hydrates, in the case of water molecules, or solvates in the case of water molecules and or organic solvents such as but not limited to ethanol may have one or more water or solvent molecules present within the crystal lattice of the compounds.

“Stereoisomers” refers to compounds having identical molecular formulae and nature or sequence of bonding but differing in the arrangement of their atoms in space. Many of the compounds of the present invention, or their pharmaceutically acceptable salts, have at least two asymmetric carbon atoms in their structure, and may therefore exist as single stereoisomers, racemates, and as mixtures of enantiomers and diastereomers. All such single stereoisomers, racemates and mixtures thereof are intended to be within the scope of this invention.

The term “mammal” includes humans and all domestic and wild animals, including, without limitation, cattle, horses, swine, sheep, goats, dogs, cats, and the like.

The phrase “therapeutically effective amount” refers to that amount of a compound of the present invention which, when administered to a mammal in need thereof, is sufficient to effect treatment, as defined below, for disease-states alleviated by the inhibition of IAP activity. The amount of a compound of the present invention which constitutes a “therapeutically effective amount” will vary depending on the compound, the disease-state and its severity, and the mammal to be treated, but can be determined routinely by one of ordinary skill in the art having regard to his own knowledge and to this disclosure.

The terms “treating” or “treatment” as used herein cover the treatment of a disease-state in a sample of cells, tissure, mammal, and particularly in a human. The disease-state in the case of over expression of IAP proteins in cells may be alleviated by the inhibition of IAP-caspase interaction and can include: preventing the disease-state from occurring in a mammal, in particular, when such mammal is predisposed to the disease-state but has not yet been diagnosed as having it; inhibiting the disease-state, i.e., arresting its development; or relieving the disease-state, i.e., causing regression of the disease-state.

The expression of IAP in cells can be detected in patients without the need for surgery. Accordingly, the present invention encompasses compounds and methods for detecting intracellular biochemical activities in living, whole animals, tissues, or cells by administering IAP binding cargo molecules of this invention which translocate into cells, and which are detectable in living cells at distances removed from the cells by the presence of intervening tissue. Examples of tissues to which the methods of the present invention can be applied include, for example, cancer cells, in particular, central nervous system tumors, breast cancer, liver cancer, lung, head and neck cancer, lymphomas, leukemias, multiple myeloma, bladder cancer, ovarian cancer, prostate cancer, renal tumors, sarcomas, colon and other gastrointestinal cancers, metastases, and melanomas. Other examples of diseases, conditions or disorders to which the present invention can be applied include, but are not limited to infection, inflammation, neurodegenerative diseases such as Alzheimer disease and Parkinson's disease.

Apoptosis may promoted in a sample of cells by administering to the cells an amount of an IAP-binding cargo molecule effective to stimulate apoptosis in the cell. The cells may be cultured cells, cells from within a tissue, and the tissue preferably is located within a living organism, preferably an animal, more preferably a mammal, and most preferably a human. These latter embodiments are carried out by formulating the IAP-binding cargo molecules of the invention as a pharmaceutical preparation for administration to a subject. Such a pharmaceutical preparation constitutes another aspect of the present invention.

The ability of a pharmaceutical agent to simulate or inhibit apoptosis is tested in a cell-free activity assay of downstream targets of IAP. In the absence of an IAP-binding cargo molecule, IAP itself interacts with and inhibits activity of caspases, thereby arresting apoptosis. Such assays include, but are not limited to, direct caspase-9 activity assays and caspase activation assays (cleavage of procaspases). In these assays, an IAP-binding cargo molecule of the invention, having a pre-determined level of activity in such assays, is used as a positive control and, optionally, a corresponding peptide known not to be active in the assay (e.g., a peptide deleted or replaced at the N-terminal Alanine) is used as a negative control. Assays are conducted using these controls, and the cells undergoing the treatment evaluated on relief of inhibition of a caspase by IAP.

The ability of a candidate drug to stimulate or inhibit apoptosis in a cultured cell is tested, according to standard methods. In these assays, an IAP-binding cargo molecule of the invention, having a pre-determined level of activity in such assays, is used as a positive control and, optionally, a corresponding peptide known not to be active in the assay (e.g., a peptide deleted or replaced at the N-terminal Alanine) is used as a negative control. Assays are conducted using these controls, and selected test compounds are tested for their ability to stimulate or inhibit apoptosis. The cells that undergo apoptosis can be differentiated from normal cells by distinct morphological changes or by molecular markers, such as cleavage of chromosomes into nucleosome ladders (detected by nuclear DNA staining).

IAP-binding cargo molecules will find utility in a wide variety of applications, some of which are listed below and others of which will be appreciated by persons of skill in the art of medical diagnostics and treatments. For instance, as described above, labeled IAP-binding cargo molecules have been used to visualize IAP expression levels in normal and altered cells, including cancer cells. The IAP-binding cargo molecules may be used in this manner to also visualize IAP in tissue samples, including biopsy, for clinical annotation. The utility of the compounds for this application has already been demonstrated for validation of “pap smear” tests.

Various aspects of the present invention will be illustrated with reference to the following non-limiting examples.

### EXAMPLE 1

This example describes the synthesis of NH₃⁺-AVPC-(badan). Unless otherwise stated, materials were purchased from Aldrich Chemical Co. (Milwaukee, Wis.) or Fisher Scientific (Pittsburgh, Pa.) and used without further purification. Methylbenzhydrylamine (MBHA) solid-phase peptide synthesis resin and Fmoc amino acids were obtained from Advanced ChemTech (Louisville, Ky.) and NovaBiochem (San Diego, Calif.). Badan dye was obtained from Molecular Probes (Eugene, Oreg.).

The peptide was synthesized on a hand shaker by Fmoc protocol on MBHA resin (Chan, W. C.; White, P. D. Fmoc Solid Phase Peptide Synthesis: A Practical Approach; Oxford University Press: Oxford, 2000). The MBHA resin was chosen because the protocol requires that it be stable under both acidic and basic conditions. The Ala-Val-Pro-Cys peptide (SEQ ID NO: 17) was synthesized using a trityl group to protect the cysteine thiol. Prior to the deprotection of the Fmoc group of the alanine, the trityl group was removed by the addition of trifluoroacetic acid (TFA), and the cysteine was derivatized with badan in the presence of diisopropylethylamine (DIEA). The Fmoc group of the alanine was removed with piperidine and then cleavage from the resin was effected by treatment with anhydrous HF containing 10% v/v anisole as scavenger at 0° C. for 45 minutes. The labeled peptide was purified by HPLC on a Vydac C18 preparative column with gradient elution by solvents A (99% H₂O; 1% CH₃CN; 0.1% TFA) and B (90% CH₃CN; 10% H₂O; 0.1% TFA) and lyophilized to dryness prior to reconstitution in H₂O. Badan, 6-bromoacetyl-2-dimethyl-aminonaphthalene, is a fluorogenic dye that may be coupled to the peptide by the methods disclosed in (Boxrud et al. J. Biol. Chem. 275: 14579-14589, 2000; Owenius et al., Biophys. J. 77: 2237-2250, 1999; Hiratsuka, T. J. Biol. Chem. 274: 29156-29163, 1999; and WO 02/096930) the contents of each are incorporated herein by reference in their entirety.

### EXAMPLE 2

This example illustrates the use of an labeled IAP-binding cargo molecule to bind with IAP within cells and tissues and inducing apoptosis.

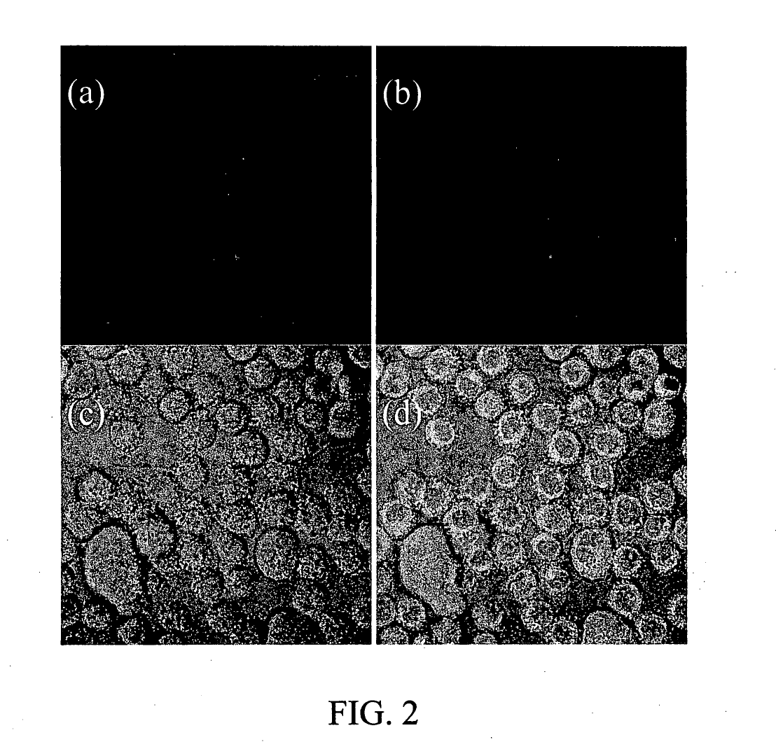

FIG. 2 shows confocal microscope images of HeLa cells loaded with AVPC-badan (17 mM) after 48 minutes; (a) emission observed between 385-470 nm; (b) emission observed with a 505-550 nm bandpass filter; (c) transmitted white light image; and (d) composite of a, b and c. AVPC-badan induced apoptosis with kinetics similar to those of the Smac N-terminal tetrapeptide AVPI (SEQ ID NO: 18) were observed. Of particular significance, the dye label enabled viewing of the localization of the peptide in a diffuse cytosolic distribution, consistent with the localization of the XIAP (SEQ ID NO: 1) target (FIG. 2). Moreover, the observed emission was uniquely characteristic of AVPC-badan binding to the XIAP BIR3 domain (SEQ ID NO: 15) (between 435-485 nm). The ability of labeled IAP-binding cargo molecules to behave in the same way provides a novel method for direct imaging and targeting of an important oncogenic molecule (IAPs) in situ in living cells.

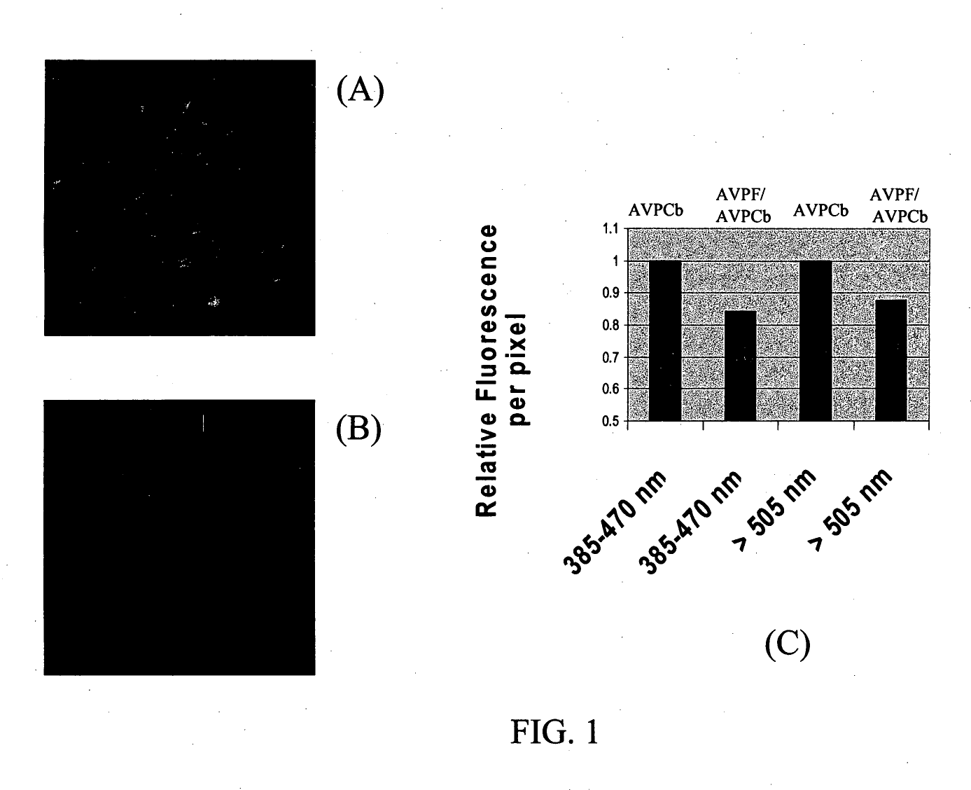

When this emission was monitored in the presence of a competitor peptide AVPF (SEQ ID NO: 19), the intensity of emission was found to diminish in the presence of the competitor FIG. 1(A-C). Thus, the primary action of the IAP binding cargo molecule appears to be by binding to IAP proteins, thus releasing any activated caspases and thereby promoting apoptosis.

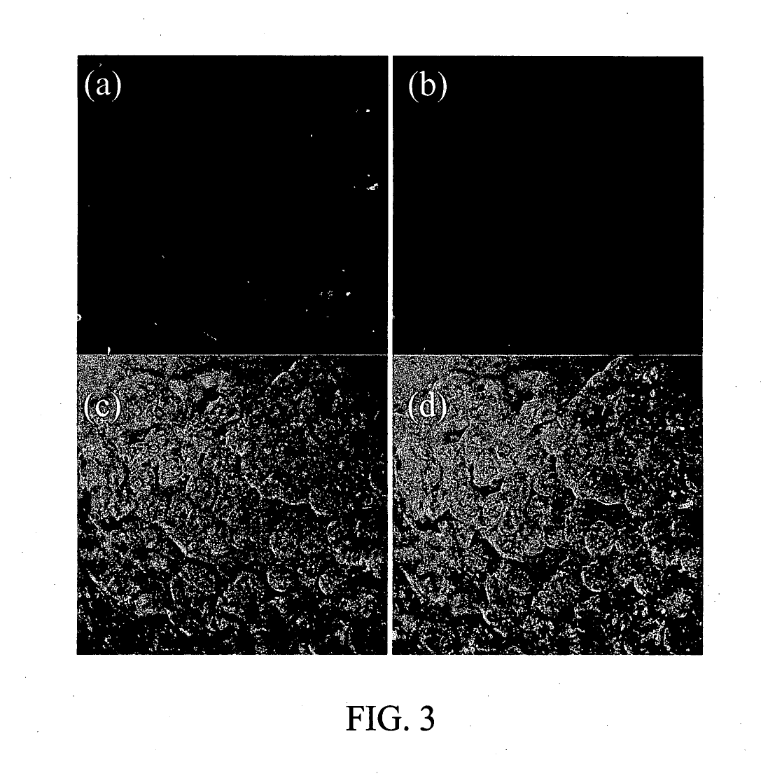

IAPs are expressed in neoplastic cells and this expression provides a reliable marker of cancer cells in situ. IAP-binding cargo molecules may be utilized for direct detection of IAP overexpression in cancer cells, and thus distinguish neoplastic from normal cells. This is illustrated by comparison of FIG. 2 and FIG. 3. FIG. 3 shows confocal microscope images of MCF7 cells loaded with AVPC-badan (17 mM) after 48 minutes; (a) emission observed between 385-470 nm; (b) emission observed with a 505-550 nm bandpass filter; (c) transmitted white light image; and (d) composite of a, b and c. In FIG. 3 (d), the contrast between the background region and the cytosol loaded with AVPC-badan is minimal in MCF7 cells, where XIAP (SEQ ID NO: 1) expression is low. Where a population of HeLa cells (high XIAP (SEQ ID NO: 1) production) and MCF7 cells (low XIAP production) are treated with AVPC-badan and compared via confocal microscopy, the HeLa cell population exhibits high fluorescence-related contrast in a spectral region consistent with AVPC-badan binding to XIAP (SEQ ID NO: 1) (FIG. 2), whereas the MCF7 cell population exhibits low contrast. This type of comparison may be used to identify overexpression of IAP in cells and may also be used to monitor the progress of a treatment regiment directed to cells and modify IAP expression.

The utility of the labeled IAP-binding cargo molecules as reliable markers to distinguish high IAP-producing from normal cells in situ has also been demonstrated in the following cell lines: A498 (renal cancer), MDA-MB-45 (breast), HeLa (cervical) (these three lines producing high levels of IAP), MCF7 (breast), NCI/ADR-RES (breast), IMR90 (normal lung fibroblasts) (these latter three lines producing low levels of IAP). Thus, the approach of using IAP-binding cargo molecules to localize neoplastic cells may be applied to a diversity of cell types.

### EXAMPLE 3

In this prophetic example, a method and composition for detecting an IAP protein in a cell is described. The method includes contacting the cell with an IAP protein detecting effective amount of a compound that has a cell membrane-permeant tetrapeptide ALPI (SEQ ID NO: 25) binding portion with labeled leucine amino acid as the diagnostic Carbon-11 radionuclide in the cargo portion of the compound.

The radionuclide labeled IAP binding cargo molecule can be use for diagnosing the presence of a disease, condition, or disorder in an mammal. Administering to the to the mammal a diagnostically effective amount of the A⁽¹¹⁾LPI (SEQ ID NO:87) IAP binding cargo molecule with ¹¹C leucine amino acid in the peptide in a pharmaceutically acceptable excipient. The disease, condition, or disorder can be a cancer such as a central nervous system tumor, breast cancer, liver cancer, lung cancer, head cancer, neck cancer, a lymphoma, or a melanoma.

The ALPI IAP binding cargo molecule with ¹¹C leucine amino acid in the peptide provides a method of assessing the effectiveness of cancer therapy. By administering a diagnostically effective amount of A⁽¹¹⁾LPI (SEQ ID NO: 87) IAP binding cargo molecule with ¹¹C leucine, the location of the IAP binding cargo molecule can be determined and a quantitative assessment of the amount of IAP binding cargo molecule in the sample made. After a course of treatment, which may involve the use of the labeled A⁽¹¹⁾LPI (SEQ ID NO: 87) to induce apoptosis, the amount of A⁽¹¹⁾LPI (SEQ ID NO: 87) IAP binding cargo molecule with ¹¹C leucine would be expected to change; a decrease in positron emission indicating fewer cells expressing high levels of IAP. Such monitoring can be performed quantitatively. Furthermore, the method can be repeated at intervals during the cancer therapy, and the quantity of the diagnostic substance detected within the mammal at each interval can be compared to the quantity of the diagnostic substance detected at previous intervals to determine the effectiveness of the therapy.

Although the present invention has been described in considerable detail with reference to certain preferred embodiments thereof, other versions are possible. Therefore the spirit and scope of the appended claims should not be limited to the description and the preferred versions contain within this specification.

1. A composition comprising:
cells; and
an IAP binding cargo molecule, said IAP binding cargo molecule having a detectable property which is modified upon binding of the molecule to IAP in the cells.

2. The composition of claim 1 wherein the detectable property is emission of light.

3. The composition of claim 1 wherein said IAP bonding cargo molecule is peptide of formula (IV)
Xₐₐ₁(Xₐₐ)_{Y}X_{aa(Y+1)}-cargo (IV)
Xₐₐ₁ is Ala or Abu, and wherein said cargo portion is fluorogenic.

4. The composition of claim 1 wherein said cell are from a bodily fluid, tissue, or combination of these.

5. The composition of claim 1 wherein said cells include neoplasitic cells.

6. The composition of claim 3 wherein the cargo portion of the molecule is badan.

7. The composition of claim 1 wherein said IAP binding cargo molecule binds to the BIR3 (SEO ID NO: 15) surface groove of an IAP.

8. The composition of claim 1 wherein said IAP binding cargo molecule displaces IAP from a caspase in said cells.

9. The composition of claim 1 wherein said IAP bonding cargo molecule is an NNR-active nucleus or MRI constast agent and the selective identification is performed through nuclear magnetic resonance or magnetic resonance imaging.

10. The composition of claim 1 wherein said IAP bonding cargo molecule is AVPC-badan.

11. A method of identifying IAP in cells comprising:
monitoring a mixture of one or more IAP binding cargo molecules with one or more sample cells for a change in a detectable property of one or more of said IAP binding cargo molecules, said detectable property changed upon formation of a complex between the IAP binding molecule and IAP in said sample cells.

12. The method of claim 11 further comprising the act of:
comparing a change in a detectable property of one or more IAP binding cargo molecules mixed with one or more control cells to the detectable change of the one or more IAP binding cargo molecules mixed with one or more sample cells, said comparison related to the amount of IAP in said sample cells.

13. The method of claim 11 further including the act of combining one or more IAP binding cargo molecules with one or more sample cells.

14. The method of claim 11 wherein the monitoring includes absorption or emission of radiant energy by said mixture.

15. The method of claim 11 wherein the change in a detectable property of one or more of said IAP binding cargo molecules is a fluorescent emission of said IAP binding molecule.

16. The method of claim 11 wherein said IAP binding cargo molecule is capable of displacing IAP from a caspase in said sample cells.

17. The method of claim 11 wherein the IAP bonding cargo molecule is AVPC-badan.

18. A method of treating cells comprising:
identifying cells having abnormal expression of IAP in a combination of one or more sample cells with one or more IAP binding cargo molecule; and
administering an amount of the IAP-binding cargo molecule to said cells to reduce the amount of IAP in said sample cells.

19. The method of claim 18 wherein the act of identifying includes monitoring a mixture of one or more IAP binding cargo molecules with one or more sample cells for a change in a detectable property of one or more of said IAP binding cargo molecules, said detectable property changed upon formation of a complex between the IAP binding molecule and IAP in said sample cells.

20. A article comprising:
packaging material containing an IAP binding cargo composition; said packaging material including a label that indicates that the IAP binding cargo composition can be used for detecting IAP in a sample of one or more cells.

21. A method of selectively identifying neoplastic cells in a mixed population of cells, the method comprising:
contacting a sample of the mixed cell population with an IAP-binding cargo molecule under conditions enabling the IAP-binding cargo molecule to bind IAP within the neoplastic cells, thereby selectively identifying the neoplastic cells.

22. The method of claim 21, wherein the cells are cultured cells.

23. The method of claim 21, wherein the cells are removed from a subject by biopsy.

24. The method of claim 21, wherein the contacting is performed by introducing the labeled IAP-binding cargo molecule into a living subject possessing or suspected of possessing the neoplastic cells.

25. The method of claim 21, wherein the IAP-binding cargo molecule comprises a fluorogenic dye label.

26. The method of claim 25, wherein the IAP binding cargo molecule is AVPC-badan.

27. The method of claim 21, wherein the labeled IAP-binding cargo molecule comprises an NMR-active nucleus and the selective identification is performed through nuclear magnetic resonance or magnetic resonance imaging.

28. The method of claim 21, wherein the labeled IAP-binding cargo molecule comprises a contrast agent and the selective identification is performed through magnetic resonance imaging.

29. The method of claim 21, wherein the labeled IAP-binding cargo molecule comprises a radioisotope and the selective identification is performed through positron emission tomography.

30. A method of selectively damaging or killing neoplastic cells in a mixed population of cells, the method comprising:
contacting a sample of the mixed cell population with an IAP-binding cargo molecule linked to an agent that is directly or indirectly toxic to cells under conditions enabling the IAP-binding cargo molecule to bind IAP within the neoplastic cells, whereupon the agent directly or indirectly exerts its toxic effect, thereby damaging or killing the neoplastic cells.

31. The method of claim 30, wherein the agent is a radioisotope.

32. The method of claim 30, wherein the agent is a photosensitizing agent and the selective damaging or killing is performed by exposing the cell population to light.


SEQUENCE LISTING (87 sequences)

SEQ ID NO: 1 - protein, 497 aa, Homo sapiens
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGGLTDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 2 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 3 - protein, 298 aa, Homo sapiens
MGPKDSAKCLHRGPQPSHWAAGDGPTQERCGPRSLGSPVLGLDTCRAWDHVDGQILGQLRPLTEEEEEEGAGATLSRGPAFPGMGSEELRLASFYDWPLTAEVPPELLAAAGFFHTGHQDKVRCFFCYGGLQSWKRGDDPWTEHAKWFPSCQFLLRSKGRDFVHSVQETHSQLLGSWDPWEEPEDAAPVAPSVPASGYPELPTPRREVQSESAQEPGGVSPAEAQRAWWVLEPPGARDVEAQLRRLQEERTCKVCLDRAVSIVFVPCGHLVCAECAPGLQLCPICRAPVRSRVRTFLS

SEQ ID NO: 4 - protein, 618 aa, Homo sapiens
MHKTASQRLFPGPSYQNIKSIMEDSTILSDWTNSNKQKMKYDFSCELYRMSTYSTFPAGVPVSERSLARAGFYYTGVNDKVKCFCCGLMLDNWKLGDSPIQKHKQLYPSCSFIQNLVSASLGSTSKNTSPMRNSFAHSLSPTLEHSSLFSGSYSSLSPNPLNSRAVEDISSSRTNPYSYAMSTEEARFLTYHMWPLTFLSPSELARAGFYYIGPGDRVACFACGGKLSNWEPKDDAMSEHRRHFPNCPFLENSLETLRFSISNLSMQTHAARMRTFMYWPSSVPVQPEQLASAGFYYVGRNDDVKCFCCDGGLRCWESGDDPWVEHAKWFPRCEFLIRMKGQEFVDEIQGRYPHLLEQLLSTSDTTGEENADPPIIHFGPGESSSEDAVMMNTPVVKSALEMGFNRDLVKQTVQSKILTTGENYKTVNDIVSALLNAEDEKREEEKEKQAEEMASDDLSLIRKNRMALFQQLTCVLPILDNLLKANVINKQEHDIIKQKTQIPLQARELIDTILVKGNAAANIFKNCLKEIDSTLYKNLFVDKNMKYIPTEDVSGLSLEEQLRRLQEERTCKVCMDKEVSVVFIPCGHLVVCQECAPSLRKCPICRGIIKGTVRTFLS

SEQ ID NO: 5 - protein, 604 aa, Homo sapiens
MNIVENSIFLSNLMKSANTFELKYDLSCELYRMSTYSTFPAGVPVSERSLARAGFYYTGVNDKVKCFCCGLMLDNWKRGDSPTEKHKKLYPSCRFVQSLNSVNNLEATSQPTFPSSVTNSTHSLLPGTENSGYFRGSYSNSPSNPVNSRANQDFSALMRSSYHCAMNNENARLLTFQTWPLTFLSPTDLAKAGFYYIGPGDRVACFACGGKLSNWEPKDNAMSEHLRHFPKCPFIENQLQDTSRYTVSNLSMQTHAARFKTFFNWPSSVLVNPEQLASAGFYYVGNSDDVKCFCCDGGLRCWESGDDPWVQHAKWFPRCEYLIRIKGQEFIRQVQASYPHLLEQLLSTSDSPGDENAESSIIHFEPGEDHSEDAIMMNTPVINAAVEMGFSRSLVKQTVQRKILATGENYRLVNDLVLDLLNAEDEIREEERERATEEKESNDLLLIRKNRMALFQHLTCVIPILDSLLTAGIINEQEHDVIKQKTQTSLQARELIDTILVKGNIAATVFRNSLQEAEAVLYEHLFVQQDIKYIPTEDVSDLPVEEQLRRLQEERTCKVCMDKEVSIVFIPCGHLVVCKDCAPSLRKCPICRSTIKGTVRTFLS

SEQ ID NO: 6 - protein, 239 aa, Homo sapiens
MAALKSWLSRSVTSFFRYRQCLCVPVVANFKKRCFSELIRPWHKTVTIGFGVTLCAVPIAQKSEPHSLSSEALMRRAVSLVTDSTSTFLSQTTYALIEAITEYTKAVYTLTSLYRQYTSLLGKMNSEEEDEVWQVIIGARAEMTSKHQEYLKLETTWMTAVGLSEMAAEAAYQTGADQASITARNHIQLVKLQVEEVHQLSRKAETKLAEAQIEELRQKTQEEGEERAESEQEAYLRED

SEQ ID NO: 7 - protein, 1248 aa, Homo sapiens
MDAKARNCLLQHREALEKDIKTSYIMDHMISDGFLTISEEEKVRNEPTQQQRAAMLIKMILKKDNDSYVSFYNALLHEGYKDLAALLHDGIPVVSSSSGKDSVSGITSYVRTVLCEGGVPQRPVVFVTRKKLVNAIQQKLSKLKGEPGWVTIHGMAGCGKSVLAAEAVRDHSLLEGCFPGGVHWVSVGKQDKSGLLMKLQNLCTRLDQDESFSQRLPLNIEEAKDRLRILMLRKHPRSLLILDDVWDSWVLKAFDSQCQILLTTRDKSVTDSVMGPKYVVPVESSLGKEKGLEILSLFVNMKKADLPEQAHSIIKECKGSPLVVSLIGALLRDFPNRWEYYLKQLQNKQFKRIRKSSSYDYEALDEAMSISVEMLREDIKDYYTDLSILQKDVKVPTKVLCILWDMETEEVEDILQEFVNKSLLFCDRNGKSFRYYLHDLQVDFLTEKNCSQLQDLHKKIITQFQRYHQPHTLSPDQEDCMYWYNFLAYHMASAKMHKELCALMFSLDWIKAKTELVGPAHLIHEFVEYRHILDEKDCAVSENFQEFLSLNGHLLGRQPFPNIVQLGLCEPETSEVYQQAKLQAKQEVDNGMLYLEWINKKNITNLSRLVVRPHTDAVYHACFSEDGQRIASCGADKTLQVFKAETGEKLLEIKAHEDEVLCCAFSTDDRFIATCSVDKKVKIWNSMTGELVHTYDEHSEQVNCCHFTNSSHHLLLATGSSDCFLKLWDLNQKECRNTMFGHTNSVNHCRFSPDDKLLASCSADGTLKLWDATSANERKSINVKQFFLNLEDPQEDMEVIVKCCSWSADGARIMVAAKNKIFLFDIHTSGLLGEIHTGHHSTIQYCDFSPQNHLAVVALSQYCVELWNTDSRSKVADCRGHLSWVHGVMFSPDGSSFLTSSDDQTIRLWETKKVCKNSAVMLKQEVDVVFQENEVMVLAVDHIRRLQLINGRTGQIDYLTEAQVSCCCLSPHLQYIAFGDENGAIEILELVNNRIFQSRFQHKKTVWHIQFTADEKTLISSSDDAEIQVWNWQLDKCIFLRGHQETVKDFRLLKNSRLLSWSFDGTVKVWNIITGNKEKDFVCHQGTVLSCDISHDATKFSSTSADKTAKIWSFDLLLPLHELRGHNGCVRCSAFSVDSTLLATGDDNGEIRIWNVSNGELLHLCAPLSEEGAATHGGWVTDLCFSPDGKMLISAGGYIKWWNVVTGESSQTFYTNGTNLKKIHVSPDFKTYVTVDNLGILYILQTLE

SEQ ID NO: 8 - protein, 416 aa, Homo sapiens
MDEADRRLLRRCRLRLVEELQVDQLWDALLSRELFRPHMIEDIQRAGSGSRRDQARQLIIDLETRGSQALPLFISCLEDTGQDMLASFLRTNRQAAKLSKPTLENLTPVVLRPEIRKPEVLRPETPRPVDIGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 9 - protein, 277 aa, Homo sapiens
MENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETDSGVDDDMACHKIPVDADFLYAYSTAPGYYSWRNSKDGSWFIQSLCAMLKQYADKLEFMHILTRVNRKVATEFESFSFDATFHAKKQIPCIVSMLTKELYFYH

SEQ ID NO: 10 - protein, 438 aa, Drosophila melanogaster
MASVVADLPSYGPIAFDQVDNNTNATQLFKNNINKTRMNDLNREETRLKTFTDWPLDWLDKRQLAQTGMYFTHAGDKVKCFFCGVEIGCWEQEDQPVPEHQRWSPNCPLLRRRTTNNVPINAEALDRILPPISYDICGANDSTLEMREHAYAEGVIPMSQLIQSIGMNAVNAAGSVTGTAAPQPRVTVATHASTATQATGDVQPETCRPSAASGNYFPQYPEYAIETARLRTFEAWPRNLKQKPHQLAEAGFFYTGVGDRVRCFSCGGGLMDWNDNDEPWEQHALWLSQCRFVKLMKGQLYIDTVAAKPVLAEEKEESTSIGGDTVASTQASEEEQQTSLSSEEAVSGDVAPSVAPTAATRIFNKIVEATAVATPSTNSSGSTSIPEEKLCKICYGAEYNTAFLPCGHVVACAKCASSVTKCPLCRKPFTDVMRVYFS

SEQ ID NO: 11 - protein, 498 aa, Drosophila melanogaster
MTELGMELESVRLATFGEWPLNAPVSAEDLVANGFFATGNWLEAECHFCHVRIDRWEYGDQVAERHRRSSPICSMVLAPNHCGNVPRSQESDNEGNSVVDSPESCSCPDLLLEANRLVTFKDWPNPNITPQALAKAGFYYLNRLDHVKCVWCNGVIAKWEKNDNAFEEHKRFFPQCPRVQMGPLIEFATGKNLDELGIQPTTLPLRPKYACVDARLRTFTDWPISNIQPASALAQAGLYYQKIGDQVRCFHCNIGLRSWQKEDEPWFEHAKWSPKCQFVLLAKGPAYVSEVLATTAANASSPPATAPAPTLQADVLMDEAPAKEALALGIDGGVVRNAIQRKLLSSGCAFSTLDELLHDIFDDAGAGAALEVREPPEPSAPFIEPCQATTSKAASVPIPVADSIPAKPQAAEAVANISKITDEIQKMSVATPNGNLSLEEENRQLKDARLCKVCLDEEVGVVFLPCGHLATCNQCAPSVANCPMCRADIKGFVRTFLS

SEQ ID NO: 12 - protein, 410 aa, Drosophila melanogaster
MAVPFYLPEGGADDVASSSSGASGNSSPHNHPLPSSASSSVSSSGVSSASASSASSSSSASSDGASSAASQSPNTTTSSATQTPMQSPLPTDQVLYALYEWVRMYQSQQSAPQIFQYPPPSPSCNFTGGDVFFPHGHPNPNSNPHPRTPRTSVSFSSGEEYNFFRQQQPQPHPSYPAPSTPQPMPPQSAPPMHCSHSYPQQSAHMMPHHSAPFGMGGTYYAGYTPPPTPNTASAGTSSSSAAFGWHGHPHSPFTSTSTPLSAPVAPKMRLQRSQSDAARRKRLTSTGEDEREYQSDHEATWDEFGDRYDNFTAGRERLQEFNGRIPPRKKKSSNSHSSSSNNPVCHTDSQPGGTSQAESGAIHGHISQQRQVERERQKAKAEKKKPQSFTWPTVVTVFVLAMGCGFFAAR

SEQ ID NO: 13 - protein, 138 aa, Drosophila melanogaster
MAIAYFIPDQAQLLARSYQQNGQQTAASPRTTATAAAPSQQQQQSQQQQQQQRHHHQQQRPQFRANISVPLGSQQGSMTMSEFGCWDLLAQIFCYALRIYSYSSSQRQPTVIQISFEISSGGQNNDEDDVTDATSKEN

SEQ ID NO: 14 - protein, 65 aa, Drosophila melanogaster
MAVAFYIPDQATLLREAEQKEQQILRLRESQWRFLATVVLETLRQYTSCHPKTGRKSGKYRKPSQ

SEQ ID NO: 15 - protein, 66 aa, Homo sapiens
YEARIFTFGTWIYSVNKEQLARAGFYALGEGDKVKCFHCGGGLTDWKPSEDPWEQHAKWYPGCKYL

SEQ ID NO: 16 - protein, 66 aa, Homo sapiens; identical to SEQ ID NO: 15

SEQ ID NO: 17 - protein, 4 aa, Artificial
  Synthetic Peptide
AVPC

SEQ ID NO: 18 - protein, 4 aa, Artificial
  Synthetic Peptide
AVPI

SEQ ID NO: 19 - protein, 4 aa, Artificial
  Synthetic Peptide
AVPF

SEQ ID NO: 20 - protein, 4 aa, Artificial
  Synthetic Peptide
ARPI

SEQ ID NO: 21 - protein, 4 aa, Artificial
  Synthetic Peptide
GVPI

SEQ ID NO: 22 - protein, 4 aa, Artificial
  SYNTHETIC PEPTIDE
AGPI

SEQ ID NO: 23 - protein, 5 aa, Artificial
  Synthetic Peptide
ABVPI

SEQ ID NO: 24 - protein, 4 aa, Artificial
  Synthetic Peptide
AVPW

SEQ ID NO: 25 - protein, 4 aa, Artificial
  Synthetic Peptide
ALPI

SEQ ID NO: 26 - protein, 5 aa, Artificial
  Synthetic Peptide
ABVPI

SEQ ID NO: 27 - protein, 4 aa, Artificial
  Synthetic Peptide
AIPI

SEQ ID NO: 28 - protein, 4 aa, Artificial
  Synthetic Peptide
AVPY

SEQ ID NO: 29 - protein, 4 aa, Artificial
  Synthetic Peptide
AHPI

SEQ ID NO: 30 - protein, 4 aa, Artificial
  Synthetic Peptide
AIPV

SEQ ID NO: 31 - protein, 4 aa, Artificial
  Synthetic Peptide
AIPY

SEQ ID NO: 32 - protein, 4 aa, Artificial
  Synthetic Peptide
AVPL

SEQ ID NO: 33 - protein, 4 aa, Artificial
  Synthetic Peptide
AVPD

SEQ ID NO: 34 - protein, 4 aa, Artificial
  Synthetic Peptide
AVPT

SEQ ID NO: 35 - protein, 4 aa, Artificial
  Synthetic Peptide
AVPV

SEQ ID NO: 36 - protein, 4 aa, Artificial
  Synthetic Peptide
AVPG

SEQ ID NO: 37 - protein, 4 aa, Artificial
  Synthetic Peptide
AVPH

SEQ ID NO: 38 - protein, 4 aa, Artificial
  Synthetic Peptide
AVPQ

SEQ ID NO: 39 - protein, 4 aa, Artificial
  Synthetic Peptide
AVPA

SEQ ID NO: 40 - protein, 4 aa, Artificial
  Synthetic Peptide
AVPM

SEQ ID NO: 41 - protein, 4 aa, Artificial
  Synthetic Peptide
AVPE

SEQ ID NO: 42 - protein, 4 aa, Artificial
  Synthetic Peptide
AVPN

SEQ ID NO: 43 - protein, 4 aa, Artificial
  Synthetic Peptide
AVPS

SEQ ID NO: 44 - protein, 4 aa, Artificial
  Synthetic Peptide
AVPP

SEQ ID NO: 45 - protein, 4 aa, Artificial
  Synthetic Peptide
AVPK

SEQ ID NO: 46 - protein, 4 aa, Artificial
  Synthetic Peptide
AVPR

SEQ ID NO: 47 - protein, 4 aa, Artificial
  Synthetic Peptide
AIPF

SEQ ID NO: 48 - protein, 4 aa, Artificial
  Synthetic Peptide
ARPF

SEQ ID NO: 49 - protein, 9 aa, Artificial
  Synthetic Peptide
AVPIAQKSE

SEQ ID NO: 50 - protein, 4 aa, Artificial
  Synthetic Peptide
AVAV

SEQ ID NO: 51 - protein, 4 aa, Artificial
  Synthetic Peptide
AVAI

SEQ ID NO: 52 - protein, 4 aa, Artificial
  Synthetic Peptide
AVAY

SEQ ID NO: 53 - protein, 4 aa, Artificial
  Synthetic Peptide
ATAV

SEQ ID NO: 54 - protein, 4 aa, Artificial
  Synthetic Peptide
ATAI

SEQ ID NO: 55 - protein, 4 aa, Artificial
  Synthetic Peptide
ATAY

SEQ ID NO: 56 - protein, 4 aa, Artificial
  Synthetic Peptide
ATAF

SEQ ID NO: 57 - protein, 4 aa, Artificial
  Synthetic Peptide
AIAV

SEQ ID NO: 58 - protein, 4 aa, Artificial
  Synthetic Peptide
AIAI

SEQ ID NO: 59 - protein, 4 aa, Artificial
  Synthetic Peptide
AVAF

SEQ ID NO: 60 - protein, 4 aa, Artificial
  Synthetic Peptide
SVPI

SEQ ID NO: 61 - protein, 4 aa, Artificial
  Synthetic Peptide
AKPI

SEQ ID NO: 62 - protein, 4 aa, Artificial
  Synthetic Peptide
AYPI

SEQ ID NO: 63 - protein, 4 aa, Artificial
  Synthetic Peptide
ACPI

SEQ ID NO: 64 - protein, 4 aa, Artificial
  Synthetic Peptide
AMPI

SEQ ID NO: 65 - protein, 4 aa, Artificial
  Synthetic Peptide
AFPI

SEQ ID NO: 66 - protein, 4 aa, Artificial
  Synthetic Peptide
AQPI

SEQ ID NO: 67 - protein, 4 aa, Artificial
  Synthetic Peptide
AWPI

SEQ ID NO: 68 - protein, 4 aa, Artificial
  Synthetic Peptide
ATPI

SEQ ID NO: 69 - protein, 4 aa, Artificial
  Synthetic Peptide
ASPI

SEQ ID NO: 70 - protein, 4 aa, Artificial
  Synthetic Peptide
ANPI

SEQ ID NO: 71 - protein, 4 aa, Artificial
  Synthetic Peptide
AEPI

SEQ ID NO: 72 - protein, 4 aa, Artificial
  Synthetic Peptide
AAPI

SEQ ID NO: 73 - protein, 4 aa, Artificial
  Synthetic Peptide
ADPI

SEQ ID NO: 74 - protein, 4 aa, Artificial
  Synthetic Peptide
APPI

SEQ ID NO: 75 - protein, 4 aa, Artificial
  Synthetic Peptide
ARPF

SEQ ID NO: 76 - protein, 4 aa, Artificial
  Synthetic Peptide
AVPF

SEQ ID NO: 77 - protein, 4 aa, Artificial
  Synthetic Peptide
AVPF

SEQ ID NO: 78 - protein, 4 aa, Artificial
  Synthetic Peptide
AVPF

SEQ ID NO: 79 - protein, 4 aa, Artificial
  Synthetic Peptide
AVPI

SEQ ID NO: 80 - protein, 4 aa, Artificial
  Synthetic Peptide
ARPI

SEQ ID NO: 81 - protein, 4 aa, Artificial
  Synthetic Peptide
AVPI

SEQ ID NO: 82 - protein, 4 aa, Artificial
  Synthetic Peptide
AIAY

SEQ ID NO: 83 - protein, 4 aa, Artificial
  Synthetic Peptide
AIAF

SEQ ID NO: 84 - protein, 4 aa, Artificial
  Synthetic Peptide
ATPY

SEQ ID NO: 85 - protein, 4 aa, Artificial
  Synthetic Peptide
ATPV

SEQ ID NO: 86 - protein, 4 aa, Artificial
  Synthetic Peptide
ATPF

SEQ ID NO: 87 - protein, 4 aa, Artificial
  Synthetic Peptide
ALPI